(19) United States
(12) Patent Application Publication
(10) Pub. No.: US 2025/0051718 A1
(43) Pub. Date: Feb. 13, 2025

(54) COST EFFECTIVE CULTURE MEDIA AND PROTOCOL FOR HUMAN INDUCED PLURIPOTENT STEM CELLS

(71) Applicant: Northwestern Unversity, Evanston, IL (US)

(72) Inventor: Paul Burridge, Evanston, IL (US)

(21) Appl. No.: 18/929,526

(22) Filed: Oct. 28, 2024

Related U.S. Application Data
(62) Division of application No. 17/025,953, filed on Sep. 18, 2020.
(60) Provisional application No. 62/902,561, filed on Sep. 19, 2019.

(51) Int. Cl. C12N 5/00; C12N 5/074

(52) U.S. Cl. CPC C12N 5/0018; C12N 5/0696; C12N 2500/60; C12N 2501/105; C12N 2501/115; C12N 2501/15; C12N 2501/33

(57) ABSTRACT

A novel culture media formula that is thoroughly optimized to support high growth rate under low seeding density conditions, require minimal media exchanges, and at low cost, while maintaining differentiation reproducibility is provided. This formula is capable of supporting both human induced pluripotent stem cell (hiPSC) generation and culture for >100 passages. Generation of B8 supplement aliquots suitable for making 100 liters of media is simple for any research lab with basic equipment, with complete bottles of media costing ˜$12 USD per liter. Weekend free hiPSC cell culture methods are possible with this formulation.

### CROSS-REFERENCE

This application claims benefit of U.S. Provisional Application No. 62/902,561, filed Sep. 19, 2019, which is incorporated herein by reference in its entirety.

### GOVERNMENT RIGHTS

This invention was made with government support under contract HL 121177 awarded by the National Institutes of Health. The Government has certain rights in the invention.

### REFERENCE TO A SEQUENCE LISTING

The instant application contains a Sequence Listing which has been filed electronically in ASCII format and is hereby incorporated by reference in its entirety. Said ASCII copy, created on Sep. 1, 2020, is named 47460-108_ST25.txt and is 16,452 bytes in size.

### TECHNICAL AREA

A cell culture medium optimized in constituents and concentrations necessary to maintain pluripotent cellular state and cell proliferation, particularly of human induced pluripotent stem cells (hiPSCs), is provided.

### BACKGROUND

Human induced pluripotent stem cells (hiPSCs) are functionally immortal and can proliferate without limit while maintaining the potential to differentiate to, hypothetically, all ˜220 cell lineages within the human body. hiPSC generation has become routine due to the simplicity of amplification of CD71⁺ blood proerythroblasts (Chou et al., 2015; Chou et al., 2011; Tan et al., 2014) or myeloid cells (Eminli et al., 2009; Staerk et al., 2010) and commercial Sendai virus-based reprogramming factor expression (Fujie et al., 2014; Fusaki et al., 2009). This simplicity has resulted in increased enthusiasm for the potential applications of hiPSC-derived cells across many fields, including regenerative medicine, disease modeling, drug discovery, and pharmacogenomics.

However, these applications require the culture of either large quantities of hiPSCs or hiPSC lines derived from large numbers of patients, and three major restrictions have become evident: 1, the cost of large-scale pluripotent cell culture, which is prohibitive for high patient-number projects; 2, the time-consuming requirement for daily media changes, which is particularly problematic for laboratories in industry; 3, inter-line variability in differentiation efficacy, which is highly dependent on pluripotent culture consistency and methodology.

### SUMMARY

Human induced pluripotent stem cell (hiPSC) culture has become routine, yet pluripotent cell media costs, frequent media changes, and reproducibility of differentiation have remained restrictive, limiting the potential for large-scale projects. Here, we describe the formulation of a novel hiPSC culture medium (B8) as a result of the exhaustive optimization of medium constituents and concentrations, establishing the necessity and relative contributions of each component to the pluripotent state and cell proliferation. B8 eliminates 97% of the costs of commercial media. The B8 formula is specifically optimized for fast growth and robustness at low seeding densities.

We demonstrated the derivation of 29 hiPSC lines in B8 as well as maintenance of pluripotency long-term, while conserving karyotype stability. This formula also allows a weekend-free feeding schedule without sacrificing growth rate or capacity for differentiation. Thus, this simple, cost-effective B8 media, will enable large hiPSC disease modeling projects such as those being performed in pharmacogenomics and large-scale cell production required for regenerative medicine. Human induced pluripotent stem cell (hiPSC) culture has become routine, yet pluripotent cell media costs, frequent media changes, and reproducibility of differentiation have remained restrictive, limiting the potential for large-scale projects. Here, we describe the formulation of a novel hiPSC culture medium (B8) as a result of the exhaustive optimization of medium constituents and concentrations, establishing the necessity and relative contributions of each component to the pluripotent state and cell proliferation.

Other methods, features and/or advantages is, or will become, apparent upon examination of the following figures and detailed description. It is intended that all such additional methods, features, and advantages be included within this description and be protected by the accompanying claims.

### BRIEF DESCRIPTIONS OF SEQUENCES

SEQ ID NO: 1 is the amino acid sequence of human FGF1:
MFNLPPGNYK KPKLLYCSNG GHFLRILPDG TVDGTRDRSD QHIQLQLSAE SVGEVYIKST
ETGQYLAMDT DGLLYGSQTP NEECLFLERL EENHYNTYIS KKHAEKNWFV
GLKKNGSCKR GPRTHYGQKA ILFLPLPVSS D
MFNLPPGNYK KPKLLYCSNG GHFLRILPDG TVDGTRDRSD PHIQLQLIAE SVGEVYIKST
ETGQYLAMDT DGLLYGSQTP NEECLFLERL EENGYNTYIS KKHAEKNWFV
GLNKNGSCKR GPRTHYGQKA ILFLPLPVSS D
SEQ ID NO: 3 is the amino acid sequence of human FGF2:
MAAGSITTLP ALPEDGGSGA FPPGHFKDPK RLYCKNGGFF LRIHPDGRVD GVREKSDPHI
KLQLQAEERG VVSIKGVCAN RYLAMKEDGR LLASKCVTDE CFFFERLESN
NYNTYRSRKY TSWYVALKRT GQYKLGSKTG PGQKAILFLP MSAKS
SEQ ID NO: 4 is the amino acid sequence of human FGF2 K128N:
MAAGSITTLP ALPEDGGSGA FPPGHFKDPK RLYCKNGGFF LRIHPDGRVD GVREKSDPHI
KLQLQAEERG VVSIKGVCAN RYLAMKEDGR LLASKCVTDE CFFFERLESN
NYNTYRSRKY TSWYVALNRT GQYKLGSKTG PGQKAILFLP MSAKS
SEQ ID NO: 5 is the amino acid sequence of human FGF2-G3 R31L, V52T, E54D,
H69F, L92Y, S94I, C96N, S109E, T121P:
MAAGSITTLP ALPEDGGSGA FPPGHFKDPK LLYCKNGGFF LRIHPDGRVD GTRDKSDPFI
KLQLQAEERG VVSIKGVCAN RYLAMKEDGR LYAIKNVTDE CFFFERLEEN NYNTYRSRKY
PSWYVALKRT GQYKLGSKTG PGQKAILFLP MSAKS
SEQ ID NO: 6 is a nucleotide sequence to generate the growth factor plasmid for
FGF1-4X:
GGATCCATGTTCAACTTACCCCCCGGCAACTACAAGAAGCCGAAGCTGCTGTATTGC
AGCAATGGCGGCCACTTTCTGCGCATTTTACCGGATGGTACCGTTGATGGTACCCGT
GATCGTTCAGATCCGCACATCCAGTTACAGCTGATCGCAGAAAGCGTGGGTGAAGT
GTACATCAAGAGCACCGAAACCGGCCAGTATCTGGCAATGGATACCGATGGCCTGC
TGTATGGTTCACAAACCCCGAACGAAGAATGCCTGTTCCTGGAACGCCTGGAAGAA
AACGGCTACAACACCTACATCAGCAAGAAGCACGCGGAGAAGAACTGGTTTGTTGG
CCTGAACAAGAACGGCAGCTGCAAACGTGGTCCTCGTACCCATTATGGCCAGAAAG
CGATTC TGTTTCTGCCGTTACCGGTTAGCAGCGATGAATTC
SEQ ID NO: 7 is a nucleotide sequence to generate the growth factor plasmid for
FGF2-K128N:
GGATCCATGGCAGCAGGTAGCATTACTACTTTACCGGCGCTGCCGGAAGATGGTGGT
TCAGGTGCATTTCCTCCTGGCCACTTCAAAGATCCTAAACGCCTGTACTGCAAGAAT
GGCGGCTTCTTTCTGCGCATTCACCCGGATGGCCGTGTTGATGGTGTTCGCGAAAAA
TCAGATCCGCACATCAAGCTGCAGTTACAGGCGGAAGAACGTGGCGTTGTGAGCAT
CAAGGGCGTTTGTGCAAACCGCTATTTAGCGATGAAAGAAGACGGCCGCCTGTTAG
CGAGCAAGTGTGTGACCGACGAATGCTTCTTCTTCGAACGCCTGGAAAGCAACAACT
ACAACACCTACCGCAGCCGCAAGTACACCAGCTGGTATGTTGCGTTAAACCGTACC
GGCCAGTACAAATTAGGCAGCAAAACCGGCCCGGGTCAGAAAGCGATTCTGTTTCT
GCCTATGAGCGCGAAGAGCTGAGAATTC
SEQ ID NO: 8 is a nucleotide sequence to generate the growth factor plasmid for
FGF2-G3:
GGATCCATGGCAGCAGGTTCGATCACTACATTACCGGCACTGCCGGAAGATGGTGG
TTCAGGTGCATTTCCTCCTGGCCACTTCAAAGACCCTAAACTGCTGTACTGCAAGAA
TGGCGGCTTCTTTCTGCGCATTCACCCGGATGGCCGTGTTGATGGTACTCGCGATAA
ATCAGATCCGTTCATCAAGCTGCAGCTGCAAGCGGAAGAACGTGGCGTGGTGAGCA
TTAAGGGCGTTTGTGCAAACCGTTATTTAGCGATGAAGGAAGACGGCCGCCTGTACG
CGATCAAGAACGTGACCGACGAATGCTTCTTCTTTGAACGCCTGGAAGAAAACAAC
TACAACACCTACCGCAGCCGCAAGTACCCGAGCTGGTATGTTGCGTTAAAGCGTACC
GGCCAGTATAAATTAGGCAGCAAAACCGGTCCGGGCCAGAAGGCGATTCTGTTTCT
GCCTATGAGCGCGAAGTCAGAATTC
SEQ ID NO: 9 is a nucleotide sequence to generate the growth factor plasmid for
NRG1:
GGATCCATGAGCCACCTTGTGAAATGCGCCGAGAAGGAGAAGACCTTTTGCGTGAA
TGGCGGCGAATGCTTCATGGTGAAGGATCTGTCAAATCCGAGCCGCTACCTGTGCAA
ATGCCCGAACGAGTTTACCGGCGATCGTTGCCAGAATTACGTTATGGCGAGCTTCTA
CAAGCACCTGGGCATCGAGTTCATGGAAGCGGAGTAAGAATTC
SEQ ID NO: 10 is a nucleotide sequence to generate the growth factor plasmid for
TGFB1:
GGATCCGCGCTGGATACCAACTATTGCTTTAGCAGCACCGAAAAAAACTGCTGCGTG
CGCCAGCTGTATATTGATTTTCGCAAAGATCTGGGCTGGAAATGGATTCATGAACCG
AAAGGCTATCATGCGAACTTTTGCCTGGGCCCGTGCCCGTATATTTGGAGCCTGGAT
ACCCAGTATAGCAAAGTGCTGGCGCTGTATAACCAGCATAACCCGGGCGCGAGCGC
GGCGCCGTGCTGCGTGCCGCAGGCGCTGGAACCGCTGCCGATTGTGTATTATGTGGG
CCGCAAACCGAAAGTGGAACAGCTGAGCAACATGATTGTGCGCAGCTGCAAATGCA
GCTGAGAATTC
SEQ ID NO: 11 is a nucleotide sequence to generate the growth factor plasmid for
TGFB1m:
GGATCCGCGCTGGATACCAACTATTGCTTTAGCAGCACCGAAAAAAACTGCTGCGTG
CGCCAGCTGTATATTGATTTTCGCAAAGATCTGGGCTGGAAATGGATTCATGAACCG
AAAGGCTATCATGCGAACTTTTGCCTGGGCCCGTGCCCGTATATTTGGAGCCTGGAT
ACCCAGTATAGCAAAGTGCTGGCGCTGTATAACCAGCATAACCCGGGCGCGAGCGC
GGCGCCGAGCTGC GTGCCGCAGGCGCTGGAACCGCTGCCGATTGTGTATT
SEQ ID NO: 12 is a nucleotide sequence to generate the growth factor plasmid for
TGFB3:
GGATCCGCGCTGGATACCAACTATTGCTTTCGCAACCTGGAAGAAAACTGCTGCGTG
CGCCCGCTGTATATTGATTTTCGCCAGGATCTGGGCTGGAAATGGGTGCATGAACCG
AAAGGCTATTATGCGAACTTTTGCAGCGGCCCGTGCCCGTATCTGCGCAGCGCGGAT
ACCACCCATAGCACCGTGCTGGGCCTGTATAACACCCTGAACCCGGAAGCGAGCGC
GAGCCCGTGCTGC GTGCCGCAGGATCTGGAACCGCTGACCATTCTG
SEQ ID NO: 13 is a nucleotide sequence to generate the growth factor plasmid for
TGFB3m:
GGATCCGCGCTGGATACCAACTATTGCTTTCGCAACCTGGAAGAAAACTGCTGCGTG
CGCCCGCTGTATATTGATTTTCGCCAGGATCTGGGCTGGAAATGGGTGCATGAACCG
AAAGGCTATTATGCGAACTTTTGCAGCGGCCCGTGCCCGTATCTGCGCAGCGCGGAT
ACCACCCATAGCACCGTGCTGGGCCTGTATAACACCCTGAACCCGGAAGCGAGCGC
GAGCCCGAGCTGCGTGCCGCAGGATCTGGAACCGCTGACCATTCTGTATTATGTGGG
CCGCACCCCGAAAGTGGAACAGCTGAGCAACATGGTGGTGAAAAGCTGCAAATGCA
GCTGAAGGGAATTC
SEQ ID NO: 14 is a FGF2 sequence with a K128N substitution:
AAGSITTLP ALPEDGGSGA FPPGHFKDPK RLYCKNGGFF LRIHPDGRVD GVREKSDPHI
KLQLQAEERG VVSIKGVCAN RYLAMKEDGR LLASKCVTDE CFFFERLESN
NYNTYRSRKY TSWYVALKRT GQYKLGSKTG PGQKAILFLP MSAKS
SEQ ID NO: 15 is a FGF2-G3 sequence:
AAGSITTLP ALPEDGGSGA FPPGHFKDPK LLYCKNGGFF LRIHPDGRVD GTRDKSDPFI
KLQLQAEERG VVSIKGVCAN RYLAMKEDGR LYAIKNVTDE CFFFERLEEN NYNTYRSRKY
PSWYVALKRT GQYKLGSKTG PGQKAILFLP MSAKS
SEQ ID NO: 16 is a TGFB3 amino acid sequence:
ALDTNYCFRN LEENCCVRPL YIDFRQDLGW KWVHEPKGYY ANFCSGPCPY LRSADTTHST
VLGLYNTLNP EASASPCCVP QDLEPLTILY YVGRTPKVEQ LSNMVVKSCK CS
SEQ ID NO: 17 is a truncated version of NRG1:
SHLVKCAEKE
KTFCVNGGECFMVKDLSNPS RYLCKCPNEF TGDRCQNYVM ASFYKHLGIE FMEAE.

### BRIEF DESCRIPTIONS OF DRAWINGS

FIG. 1. Optimization of Matrix Concentration and Comparison of Media Formulae. a, Relative growth of hiPSC on dilutions of Matrigel® (Corning®) or Cultrex® (Trevigen), Cultrex® is equivalent to Geltrex® (Gibco™).

FIG. 2A-F. Optimization of Basic Human Pluripotent Stem Cell Medium Constituents with a Short-Term Growth Assay. Results are normalized to initial medium component concentrations shown with a dark gray bar, optimizations were completed using short-term 6-day growth assay. Optimized component concentrations shown with a diagonal hash. (A) Comparison of the effect of recombinant human IGF1 LR3 (n=3) and recombinant human insulin (n=18-22) concentrations on relative growth. (B) L-ascorbic acid 2-phosphate (n=19-27). (C) Transferrin (n=2-20). (D) Sodium selenite (n=3-20). (E) FGF2-K128N (n=3-12). (F) TGFb1 (n=20-25). n=full experimental replicates, unpaired Student's T-test, *P≤0.05, ** P≤0.01, *** P≤0.005, **** P≤0.0001, n.s.=not significant.

FIG. 3A-H. Optimization of Additional Human Pluripotent Stem Cell Medium Constituents in a Short-Term Assay. E8 medium component concentrations are shown with a dark gray bar, optimizations were completed using a simple 6-day growth assay. Optimized component concentrations shown with a diagonal hash. (A) Comparison of the suitability of recombinant transferrin (10 μg ml−1) to support clonal growth with and without ROCK1/2 inhibition using Y27632 (10 μM) during the first 24 h after passage (n=3). (B) Comparison of two common ROCK 1/2 inhibitors only during first 24 h after passage on relative growth (n>5). (C) Comparison of the effect of the addition of non-essential amino acids (NEAA) and chemically defined lipids (n=5) on relative growth. (D) Fatty acid-free albumin (n=4). (E) Sodium bicarbonate (n=5). (F) pH (n=9). (G) Osmolarity (n=8). (H) FGF2-G3 (n=3). n=full experimental replicates, unpaired Student's T-test, *P≤0.05, ** P≤0.01, *** P≤0.005, **** P≤0.0001, n.s.=not significant.

FIG. 4A-B. Plasmids used to Generate Recombinant Growth Factors. (A) FGF2-K128N demonstrating dual 6xHis site for purification and thrombin cleavage site. (B) Amino acid sequences used to generate modified FGF2 plasmids.

FIG. 5A-P. Optimization of B8 Medium Constituents with a Long-Term Growth Assay. Results are normalized to initial medium component concentrations shown with a dark gray bar, optimizations were completed using long-term 5-passage, 4-day growth assay. Comparison of the effect of (A) recombinant human insulin concentration on relative growth (n=10). (B) L-ascorbic acid 2-phosphate (n=10). (C) Recombinant transferrin (n=10). (D) Sodium selenite (n=5). (E) In-house made FGF2-G3 (n=6). (G) NODAL (n=5). (H) Activin A (n=5). (I) In-house made TGFb3 after 9 passages compared to commercial TGFb1 (n=9). (J) Addition of NRG1 to 40 ng ml-1 FGF2-G3 (n>5). (K) Final B8 formula. n=full experimental replicates, unpaired Student's T-test, *P≤0.05, ** P≤0.01, *** P≤0.005, **** P≤0.0001, n.s.=not significant.

FIG. 6. DNA Sequences used to Generate Growth Factor Plasmids. Note that FGF2-G3 and TGFb3 were shown to function without the need for cleavage of N-terminus 6xHis tag/fusion proteins therefore C-terminus stop codons were also removed to read through to an additional 6xHis tag to enhance purification efficiency.

FIG. 7A-E. Qualification of B8 as Suitable for hiPSC Generation and Culture. (A) Demonstration of maintenance of pluripotency markers in 29 hiPSC lines derived in B8 assessed by flow cytometry. (B) Expression of pluripotent markers in a variety of B8-derived hiPSC lines. (C) Example G-banding karyotype analysis of four hiPSC lines derived in B8 from blood. (D) hiPSC growth at low seeding densities in B8 compared to E8 (n=8). (E) Assessment of stimulation of phospho-ERK after media had been stored at 37° C. for 2 or 7 days, comparing in-house generated FGF2-G3 to a commercial FGF2 (Peprotech). hiPSC were starved of FGF2 for 24 h then treated with the indicated media for 1 h before collection for Western blot. Total ERK was used as a loading control.

FIG. 8A-H. Optimization of Weekend-Free Passaging Schedule that is Still Compatible with Monolayer Differentiation. (A) Establishment of an optimal 4-day media change schedule. (B) 7 day passage with media change schedule. (C) 7 day passage only schedule. (D) Comparison of growth when using two 7-day weekend-free passaging schedules with or without addition of 0.5 mg ml-1 albumin (n=2). (E) Comparison the addition of varying levels of albumin (mg ml-1) to a 7-day passage and media change schedule (n=4). (F) Cardiac differentiation efficiency when using 7-day passage and media change schedule (n=5). (G) Endothelial differentiation efficiency when using 7-day passage and media change schedule (n=6). (H) (G) Endothelial differentiation efficiency when using 7-day passage and media change schedule (n=5).

### DETAILED DESCRIPTION

Demonstrated herein is a novel media formula (B8), thoroughly optimized to support high growth rate under low seeding density conditions, require minimal media exchanges, and at low cost, while maintaining differentiation reproducibility. This formula is capable of supporting both hiPSC generation and culture for >100 passages. Generation of B8 supplement aliquots suitable for making 100 liters of media is simple for any research lab with basic equipment, with complete bottles of media costing ˜$12 USD per liter.

A full protocol is provided, including detailed instructions for recombinant protein production in three simple steps. The protocol is made possible by the in-lab generation of three E. coli-expressed, codon-optimized recombinant proteins: an engineered form of fibroblast growth factor 2 (FGF2) with improved thermostability (FGF2-G3); transforming growth factor β3 (TGFβ3)—a more potent TGFβ able to be expressed in E. coli; and a derivative of neuregulin 1 (NRG1) containing the EGF-like domain. All plasmids for protein production are available through Addgene. With the commoditization of these protocols, we believe it is possible to substantially increase what is achievable with hiPSCs due to the near elimination of pluripotent cell culture costs and minimization of labor associated with cell culture.

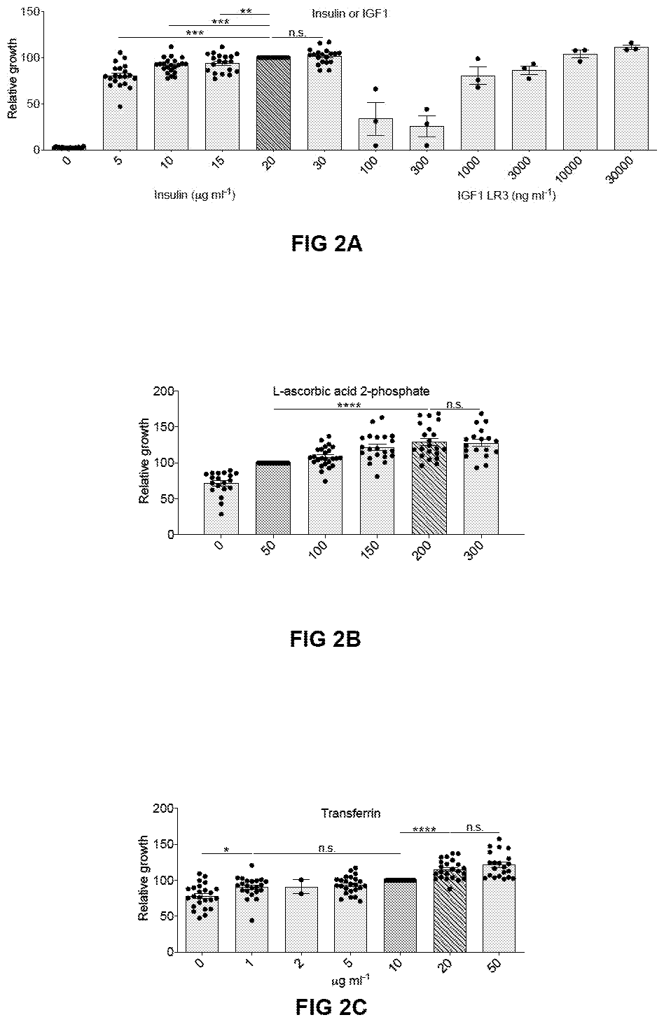

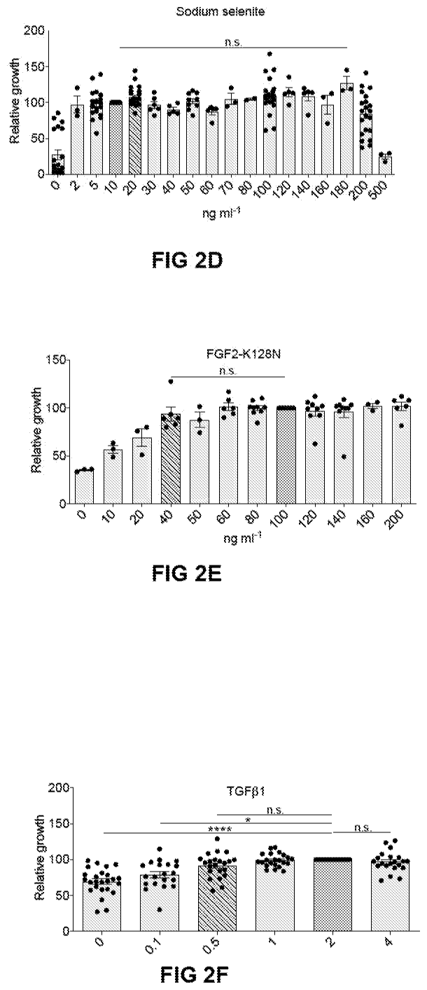

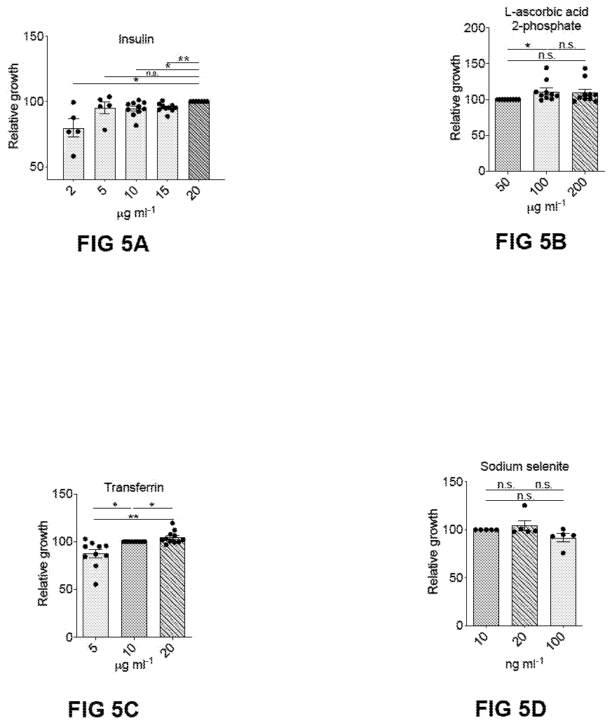

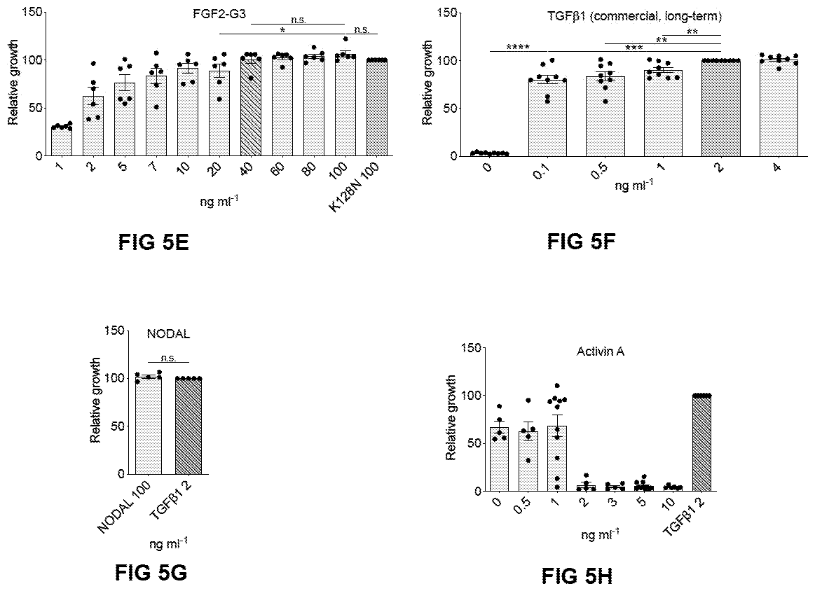

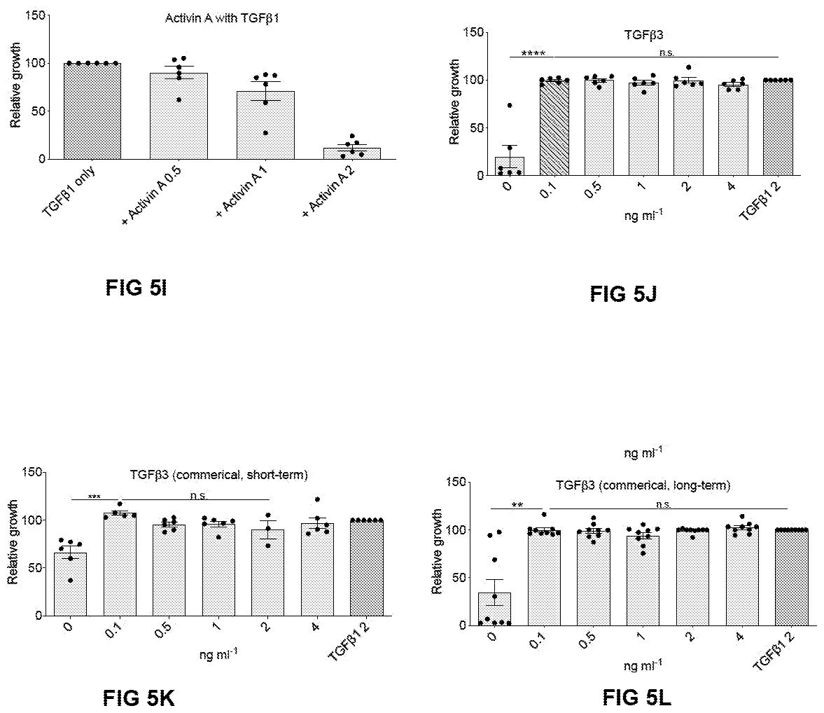

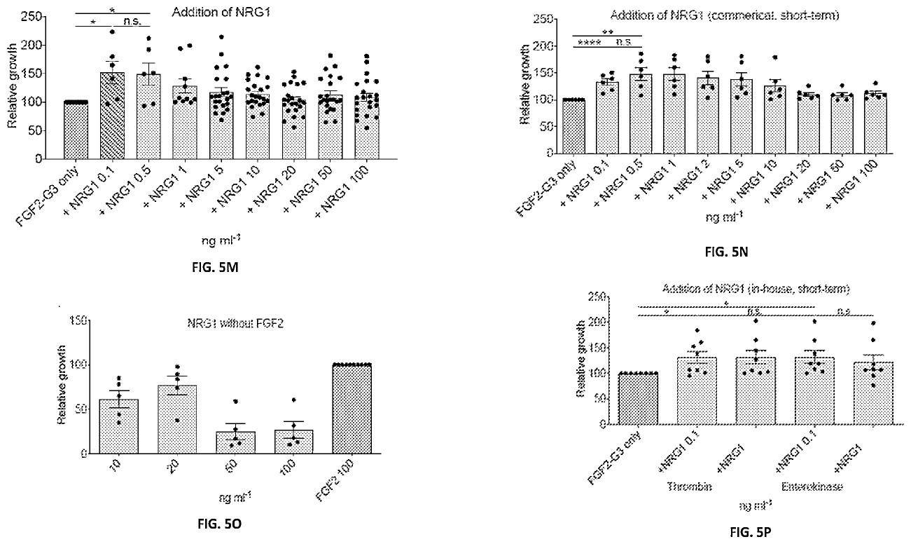

Only five components were essential for hiPSC culture: insulin, sodium selenite, FGF2, DMEM/F12, (FIG. 2) and importance of a fifth component TGFβ1 was only evident in the long-term assay (FIGS. 2 and 5). The other three components, ascorbic acid 2-phosphate, transferrin, and NRG1, are dispensable for hiPSC growth, although their removal results in a reduced growth rate.

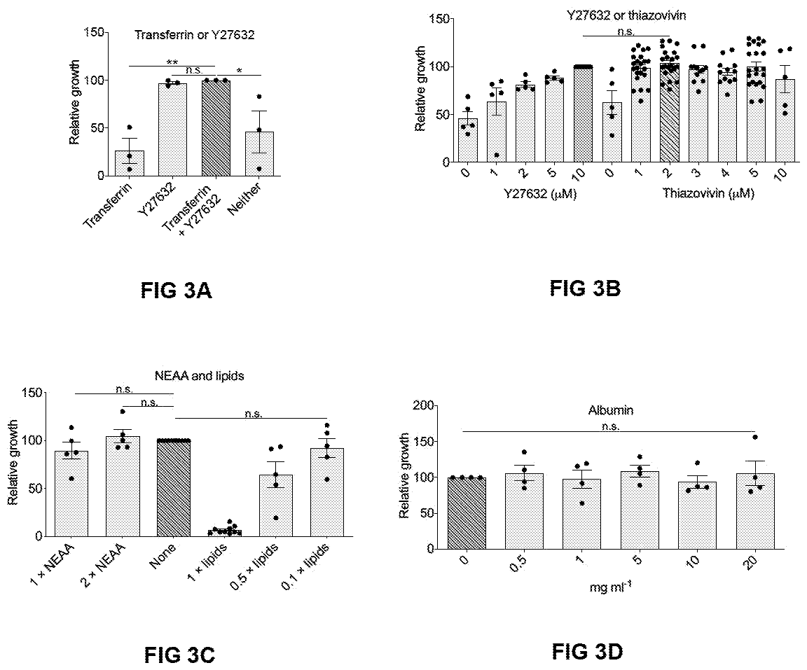

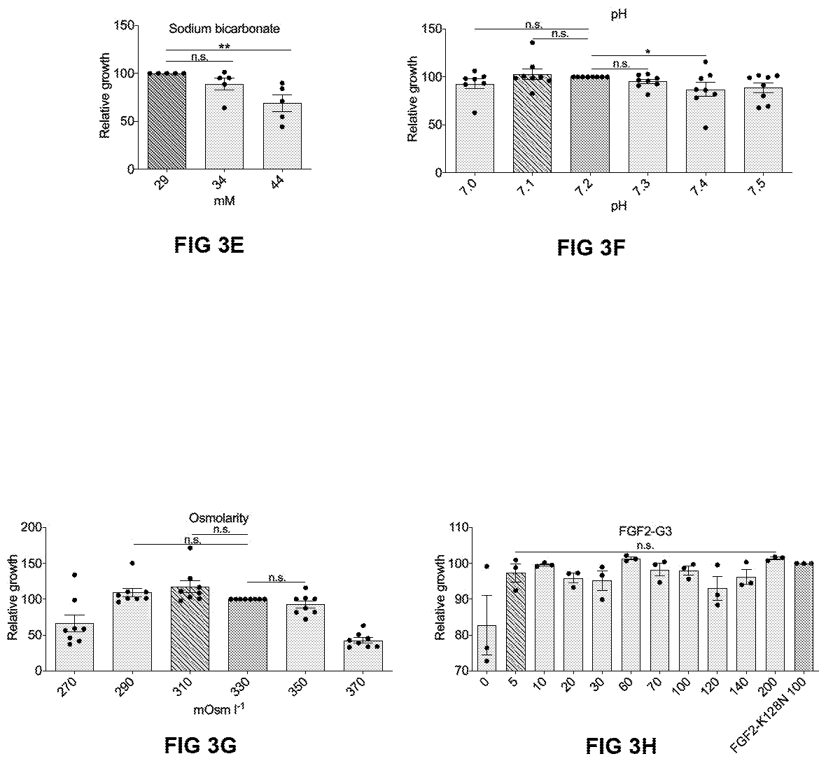

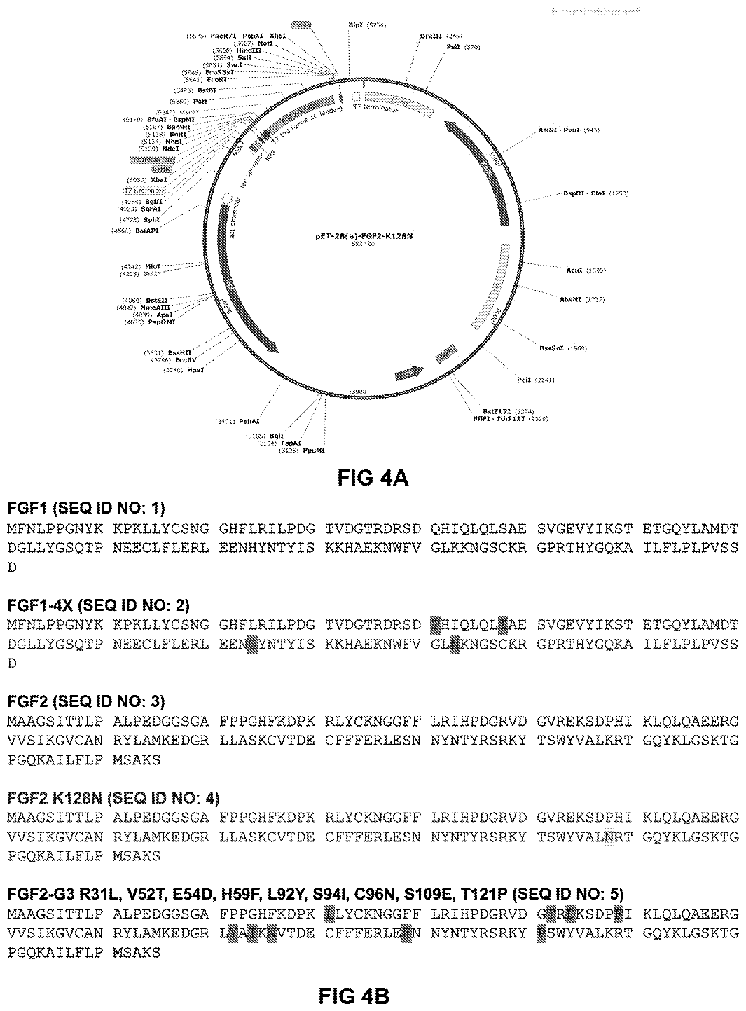

A number of surprising results in development of the culture media. For example, neither a positive or negative effect of the addition of albumin (FIG. 3D) despite it being a common constituent of many academic and commercial media formula. Activin A was not suitable either with or without TGFβ1 (FIG. 5) despite its inclusion in a variety of other commercially available formulas. We do show that at very low doses Activin A could support growth, albeit to a lesser extent than TGFβ1.

A major issue with some commercial media is that although a weekend-free schedule is feasible, growth of hiPSCs is considerably slower and it is recommended to grow cells as low-density colonies. These low-density colonies are not compatible with subsequent monolayer differentiation protocols, as have become commonplace with the majority of lineages. The optimization of the B8 culture media specifically for fast monolayer growth, along with the incorporation of thermostable FGF2-G3, overcomes many of these issues while maintaining compatibility with common differentiation protocols.

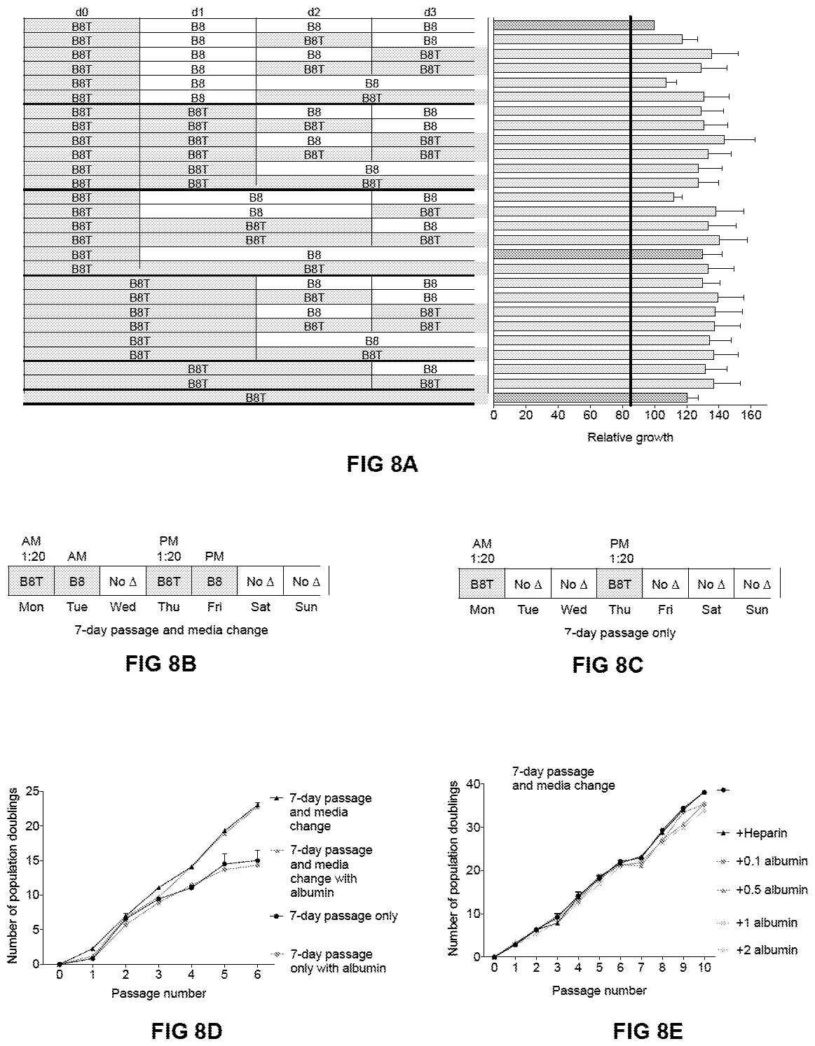

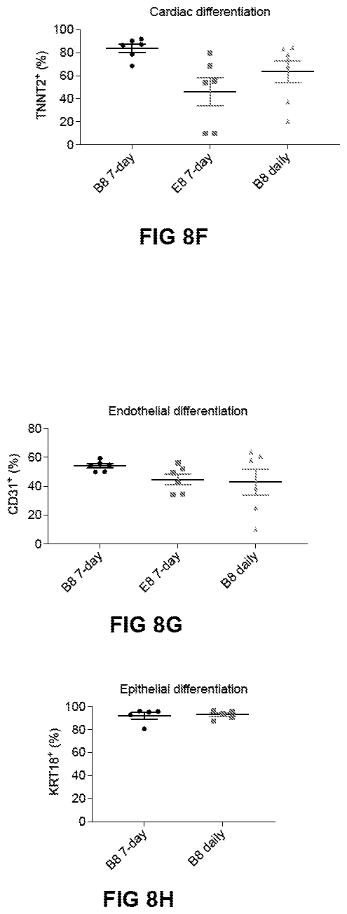

The growth factors FGF2 and TGFβ1 represented more than 80% of the total medium costs. Optimization of the plasmids and generation of thioredoxin fusion proteins where necessary eliminates much of the complexity associated with inclusion bodies and the resulting refolding processes otherwise required. A typical 1 liter E. coli culture, which requires two days and basic laboratory skills, will usually provide 80 mg of FGF2-G3, enough for 800 liters of B8. Similarly, a 500 ml culture of TGFβ3 or NRG1 will commonly provide enough protein years of work (˜800,000 liters of B8 media). Additionally, the concentrations of these components can be reduced by 75% without a substantial impact on growth rate (both at 5 μg ml⁻¹) (FIG. 5) and based on these savings, have developed a formulation of B8 optimized for low-cost (FIG. 8).

In particular, the cell medium comprises, for growth of human induced pluripotent stem cells, a cell culture base medium; a Fibroblast Growth Factor 2; insulin; and a source of selenium. The cell culture medium is not required to contain Transforming Growth Factor beta 1 (TGFβ1), Activin A, or albumin. That is the cell culture medium may contain substantially no Transforming Growth Factor beta 1 (TGFβ1), Activin A, or albumin. The cell culture medium may contain trace amounts of Transforming Growth Factor beta 1 (TGFβ1), Activin A, or albumin. The cell culture medium may contain measurable amounts of Transforming Growth Factor beta 1 (TGFβ1), Activin A, or albumin, but not in a concentration sufficient to influence cell growth, differentiation or health.

In some aspects, insulin or IGF 1 may be used in the culture media. Recombinant forms of insulin, IGF1, any derivatives or variants that are cost effective to produce without any detriment to function may be substituted for insulin or IGF1. Similarly, a source of selenium may comprise a selenium salt, L-selenomethionine, selenocysteine, methylselenocysteine or similar compounds.

In some aspects, the cell culture medium may also contain TGFβ3, NRG1; transferrin, ascorbic acid, or a combination thereof. The cell culture medium may also contain thiazovivin. The cell culture medium may be characterized by a pH of 7.1 or by an osmolarity of 310 mOsm/l. The cell culture medium may also be characterized by sodium bicarbonate in an amount of 2438 μg/ml.

In some aspects the cell culture base medium is DMEM/F12. In some aspects the FGF2 is a recombinant protein defined as either SEQ ID NO: 4, 5, or 15, or a mixture thereof. In some aspects the FGF2 is a recombinant protein FGF2-G3 (SEQ ID NO: 15). In some aspects the selenite salt is sodium selenite. In some aspects, the TGFβ3 is a recombinant protein of SEQ ID NO: 16, NRG1 is a recombinant protein of SEQ ID NO: 17. In some aspects, the cell culture medium comprises: 40 ng/ml FGF2-G3, 20 μg/ml insulin, 20 ng/ml sodium selenite, formulated in a DMEM/F12 culture medium. As an alternative, the cell culture medium may include 40 ng/ml FGF2-G3 (SEQ ID NO: 15), 20μg/ml insulin, 20 ng/ml sodium selenite, 20 μg/ml transferrin, 0.1 ng/ml TGFβ3 (SEQ ID NO: 16), 0.1 ng/ml NRG1 (SEQ ID NO: 17), 200 μg/ml ascorbic acid 2-phosphate, 2438 μg/ml sodium bicarbonate formulated in a DMEM/F12 culture medium.

Also provided herein is a kit for preparation of a cell culture medium, the kit comprising: plasmids encoding FGF2-G3, TGFβ3, and NRG1; and instructions for preparing FGF2-G3, TGFβ3, and NRG1 protein and preparing a cell culture medium. The kit may further include culture medium, sodium selenite, insulin, transferrin, ascorbic acid 2-phosphate, sodium bicarbonate, or thiazovivin.

Also provided herein are methods of growing and passing human induced pluripotent stem cells (hiPSCs) in culture, the method comprising: obtaining a cell culture medium comprising: FGF2-G3 (SEQ ID NO: 15), insulin, sodium selenite, transferrin, TGFβ3 (SEQ ID NO: 16), NRG1 (SEQ ID NO: 17), ascorbic acid 2-phosphate, sodium bicarbonate formulated in a DMEM/F12 culture medium, preparing matrix coated plates; adding hiPSCs to the matrix, day 0; changing cell culture medium on day 1; passing cells on day 3.5 or growing cells for 7 consecutive days provided that at least one day of the 3.5 day passing or the 7-day cell growth cycle will not require changing the cell culture medium.

### Base Media

The culture media described herein suggest the use of a DMEM/F12 as a culture media base. However, any appropriate culture media base can be combined with the insulin, ascorbic acid, transferrin, selenite, FGF2, TGFβ, and NRG1as described herein. In fact, Chen et al. showed comparable results between DMEM/F12 and the comparatively simple MEMα. Any other basic defined culture media may also be used in combination with the insulin, ascorbic acid, transferrin, selenite, FGF2, TGFβ, and NRG1as described herein.

### Deficiencies in Prior Art Culture Media:

Media conditions required to culture human pluripotent stem cells has progressed steadily over the last 15 years, with significant breakthroughs coming from the discovery of the necessity for high concentrations of FGF2 (Xu et al., 2005), the use of TGFβ1 (Amit et al., 2004), and the elimination of knockout serum replacement (KSR) with the TeSR formula which contains 21 components (Ludwig and Thomson, 2007; Ludwig et al., 2006a; Ludwig et al., 2006b), followed by the first robust chemically defined formula, E8 (Beers et al., 2012; Chen et al., 2011) which consists of just 8 major components. A number of alternative non-chemically defined pluripotent formulations have been described including: CDM-BSA (Hannan et al., 2013; Vallier et al., 2005; Vallier et al., 2009), DC-HAIF (Singh et al., 2012; Wang et al., 2007), hESF9T (Furue et al., 2008; Yamasaki et al., 2014), FTDA (Breckwoldt et al., 2017; Frank et al., 2012; Piccini et al., 2015), and iDEAL (Marinho et al., 2015) (FIG. S1A).

Each of the available formulations consist of a core of three major signaling components: 1) insulin or IGF1 which bind INSR and IGF1R to signal the PI3K/AKT pathway promoting survival and growth; 2) FGF2 and/or NRG1 which bind FGFR1/FGFR4 or ERBB3/ERBB4 respectively, activating the PI3K/AKT/mTOR and MAPK/ERK pathways; and 3) TGFβ1, NODAL, or activin A which bind TGFBR1/2 and/or ACVR2A/2B/1B/1C to activate the TGFβ signaling pathway. NODAL is used less commonly in pluripotent media formulations due to the expression of NODAL antagonists LEFTY1/2 in human pluripotent stem cells (hPSC) (Besser, 2004; Sato et al., 2003) resulting in a requirement for high concentrations in vitro (Chen et al., 2011). In addition, numerous growth factor-free formulae utilizing small molecules to replace some or all growth factors in hPSC culture have been described (Burton et al., 2010; Desbordes et al., 2008; Kumagai et al., 2013; Tsutsui et al., 2011), however, these have not successfully translated to common usage. Recently, a growth factor-free formula AKIT was demonstrated (Yasuda et al., 2018), combining inhibitors of GSK3B (1-azakenpaulone), DYRK1 (ID-8), and calcineurin/NFAT (tacrolimus/FK506), albeit with much reduced proliferation and colony growth, as well as increased interline variability in growth. Finally, more than 15 commercial pluripotent media are also available in which the formulae are proprietary and not disclosed to researchers. These media represent the major cost for most hiPSC labs and considerably restrict research efforts. Some of these media formulae are suggested to support hiPSC growth without daily media changes or ‘weekend-free’, likely by using heparin sulfate to stabilize FGF2 that otherwise degrades quickly at 37° C. (Chen et al., 2012; Furue et al., 2008) and including bovine serum albumin (BSA) which acts as a multifaceted antioxidant.

### EXAMPLES

Certain embodiments are described below in the form of examples. While the embodiments are described in considerable detail, it is not the intention to restrict or in any way limit the scope of the appended claims to such detail, or to any particular embodiment. The following Experimental Procedures are used throughout the Examples.

### Experimental Procedures:

### Human Induced Pluripotent Cell Culture

All pluripotent and reprogramming cell cultures were maintained at 37° C. in Heracell™ VIOS 160i direct heat humidified incubators (Thermo Scientific™) with 5% CO₂ and 5% O₂. Differentiation cultures were maintained at 5% CO₂ and atmospheric (˜21%) O₂. All cultures (pluripotent and differentiation) were maintained with 2 ml medium per 9.6 cm² of surface area or equivalent. All media was used at 4° C. and was not warmed to 37° C. before adding to cells due to concerns of the thermostability of the FGF2 (Chen et al., 2012). We have found no detectable effects on cell growth from using cold media. All cultures were routinely tested for mycoplasma using a MycoAlert™ PLUS Kit (Lonza) and a 384-well Varioskan™ LUX (Thermo Scientific™) plate reader. E8 medium was made in-house as previously described (Burridge et al., 2015; Chen et al., 2011) and consisted of DMEM/F12 (Corning®, 10-092-CM), 20 μg ml⁻¹ E. coli-derived recombinant human insulin (Gibco™, A11382IJ), 64 μg ml⁻¹ L-ascorbic acid 2-phosphate trisodium salt (Wako, 321-44823), 10 μg ml⁻¹ Oryza sativa-derived recombinant human transferrin (Optiferrin, In Vitria, 777TRF029-10G,), 14 ng ml⁻¹ sodium selenite (Sigma, S5261), 100 ng ml⁻¹ recombinant human FGF2-K128N (made in-house, see below), 2 ng ml⁻¹ recombinant human TGFβ1 (112 amino acid, HEK293-derived, Peprotech, 100-21). Cells were routinely maintained in E8 medium on 1:800 diluted growth factor reduced Matrigel® (see below). E8 was supplemented with 10 μM Y27632 dihydrochloride (LC Labs, Y-5301), hereafter referred to as E8Y, for the first 24 h after passage. For standard culture, cells were passaged at a ratio of 1:20 every 4 days using 0.5 mM EDTA (Invitrogen UltraPure) in DPBS (without Ca²⁺ and Mg²⁺, Corning®), after achieving ˜70-80% confluence. Cell lines were used between passages 20 and 100. Other media components tested were: Human Long R3 IGF1 (Sigma, 91590C), thiazovivin (LC Labs, T-9753), recombinant human TGFβ3 (Cell Guidance Systems, GFH109), sodium bicarbonate (Sigma), NEAA (Gibco™), CD Lipids (Gibco™), fatty acid-free albumin (GenDEPOT, A0100). pH was adjusted with 10N HCl or 1N NaOH (both from Sigma) and measured using a SevenCompact™ pH meter (MettlerToledo). Osmolarity was adjusted with sodium chloride (Sigma) or cell culture water (Corning®) and measured with an osmometer (Advanced Instruments).

### Matrigel® Optimization

Our standard condition throughout was 2 ml of 1:800 reduced growth factor Matrigel® (Corning®, 354230) diluted in 2 ml of DMEM (Corning®, 10-017-CV) per well of 6-well plate or equivalent. Also tested were Geltrex® (Gibco™) and Cultrex® (Trevigen). Plates were made and kept in incubators at 37° C. for up to one month.

### FGF2-K128N Generation

The full length (154 amino acid) FGF2 sequence (SEQ ID NO: 14) AAGSITTLP ALPEDGGSGA FPPGHFKDPK RLYCKNGGFF LRIHPDGRVD GVREKSDPHI KLQLQAEERG VVSIKGVCAN RYLAMKEDGR LLASKCVTDE CFFFERLESN NYNTYRSKY TSWYVALKRT GQYKLGSKTG PGQKAILFLP MSAKS with a K128N substitution (in bold/underlined) was codon optimized for E. coli with the addition of a BamHI site at the start (5′) of the sequence and an EcoRI site at the end (3′). This sequence was synthesized on a BioXp 3200 (Synthetic Genomics). The insert was then digested with BamHI and EcoRI (Anza, Invitrogen) and ligated with T4 DNA ligase (Anza) in to a pET-28a expression vector (Novagen/MilliporeSigma) and cloned in to One Shot™ BL21 Star™ (DE3) chemically competent E. coli (Invitrogen). E. coli were stored in 25% glycerol (Ultrapure, Invitrogen) at −80° C. A starter cultured was prepared by inoculating 10 ml of Terrific Broth (Fisher BioReagents) supplemented with 50 μg ml⁻¹ kanamycin sulfate (Fisher BioReagents) in a bacterial tube (Corning® Falcon) and incubated in an Innova®−44 Incubator-Shaker (New Brunswick™) at 220 rpm overnight at 37° C. (for NRG1) or 30° C. (for FGF2 or TGFβ3). Protein expression was performed using a 2800 ml baffled shaker flask (BBV2800, Fisherbrand™) as follows: The whole 10 ml starter culture was added to 500 ml of MagicMedia™ (K6815, Invitrogen™), supplemented with 50 μg/ml kanamycin sulfate and incubated as above for 24 h at 37° C. (for NRG1) or 30° C. (for FGF2 or TGFβ3). The culture was harvested in to 2×250 ml centrifuge bottles (Nalgene®, 3120-0250) and centrifuged in an Optimia™ XPN-100 ultra centrifuge (Beckman Coulter) with a SW 32 Ti rotor at 5,000×g for 20 min at 4° C. Supernatant was carefully poured off and pellets were weighed and stored at −80° C. for downstream processing. Cells pellets were resuspended B-PER lysis buffer (Thermo Scientific™, 78248) using 5 ml of B-PER Complete Reagent per gram of bacterial cell pellet. Cells were incubated for 15 minutes at RT with gentle rocking. The bottles containing the lysates were then centrifuged in an ultracentrifuge at 16,000×g for 20 min at 4° C. Supernatants were collected and the cell debris was discarded. Purification was completed using a 3 ml HisPur™ Ni-NTA Spin Purification kit (Thermo Scientific™, 88229) following the manufacturers recommendations. To enhance the protein binding efficiency to the resin bed, the sample was incubated for 30 min at 4° C. Four elutes were collected, one every 10 min. The columns were reused following the manufacturer's regeneration protocol. The protein concentration was evaluated using Quant-iT™ Qubit® Protein Assay Kit (Invitrogen, Q3321) on a Qubit 3 fluorometer. The 6xHis tag was not cleaved as it has been previously demonstrated to not interfere with the FGF2 function (Soleyman et al., 2016). A standard 1 liter culture produced 80 mg of FGF2.

### FGF2-G3 Generation

154 amino acid sequence (SEQ ID NO: 15): AAGSITTLP ALPEDGGSGA FPPGHFKDPK LLYCKNGGFF LRIHPDGR VD GTRDKSDPFI KLQLQAEERG VVSIKGVCAN RYLAMKEDGR LYAIKNVTDE CFFFERLEEN NYNTYRSRKY PSWYVALKRT GQYKLGSKTG PGQKAILFLP MSAKS with R31L, V52T, E54D, H59F, L92Y, S94I, C96N, S109E, and T121P substitutions (in bold) was codon optimized for E. coli was generated as above. FGF1-4X was similarly generated.

### TGFβ3 Generation

112 amino acid sequence (SEQ ID NO: 16): ALDTNYCFRN LEENCCVRPL YIDFRQDLGW KWVHEPKGYY ANFCSGPCPY LRSADTTHST VLGLYNTLNP EASASPCCVP QDLEPLTILY YVGRTPKVEQ LSNMVVKSCK CS was codon optimized for E. coli and generated as above, ligated in to a pET-32a expression vector, and cloned in to One Shot™ BL21 Star™ (DE3). The use of pET-32a results in the production of a thioredoxin-TGFβ3 fusion protein which prevents protein expression in inclusion bodies. It is not necessary to cleave the thioredoxin for TGFBB3 to be active. TGFβ1, TGFβ1m (C77S), and TGFβ3m (C77S) were similarly generated.

### NRG1 Generation

65 amino acid sequence (SEQ ID NO: 17): SHLVKCAEKE KTFCVNGGEC FMVKDLSNPS RYLCKCPNEF TGDRCQNYVM ASFYKHLGIE FMEAE, which is a truncated version of NRG1 containing just the EGF domain, was codon optimized for E. coli and generated as above, ligated in to a pET-32a expression vector, and cloned in to One Shot™ BL21 Star™ (DE3). The use of pET-32a results in the production of a thioredoxin-NRG1 fusion protein which prevents protein expression in inclusion bodies. It was found necessary to cleave the thioredoxin using Thrombin CleanCleave™ Kit (MilliporeSigma), followed by repurification, keeping the supernatant.

### Media Variable Optimization Protocol

The hiPSC line 19-3 was dissociated with TrypLE (Gibco™, 12604-013) for 3 min at 37° C. and cells were resuspended in DMEM/F12, transferred to a 15 ml conical tube (Falcon) and centrifuged at 200×g for 3 min (Sorvall ST40). The pellet was resuspended in DMEM/F12, diluted to 1×10⁵ cells per ml and 10,000 cells were plated per well in Matrigel® (1:800)-coated 12-well plates (Greiner) in the medium to be tested along with 2 μM thiazovivin for the first 24 h. Media were changed daily and cells were grown for 6 days. This lower than normal seeding density was used to allow the discovery of factors only detectable under more extreme conditions and therefore provide data on the robustness to the formulation.

### Western Blot

Stock lysis buffer was prepared as 150 mM NaCl, 20 mM Tris pH 7.5, 1 mM EDTA, 1 mM EGTA, and 1% Triton X-100 and stored at 4° C. Fresh complete lysis buffer was prepared with final concentration of 1× Protease Inhibitor (Roche, 5892791001), 1× Phosphatase Inhibitor Cocktail 2 (P5726, Sigma), 1× Phosphatase Inhibitor Cocktail 3 (Sigma, P0044), 2 mM PMSF (Sigma, P7626), and 1% SDS Solution (Fisher Scientific, BP2436200). hiPSC were starved using B8 without FGF2 for 24 hours, then treated with media containing the corresponding FGF2 for 1 h. Media was then removed, and cells were washed once with DPBS, harvested with 0.5 mM EDTA in DPBS, and transferred into tubes. Samples were pelleted by centrifugation at 500×g for 3 minutes and the supernatant was discarded. The pellet was resuspend in 150 μl complete lysis buffer and incubated on ice for 30 min. Clear lysates were collected by centrifugation at 10,000×g for 10 min at 4° C. The protein concentration was measured with Qubit Protein Assay Kit (Invitrogen, Q33211) and Qubit 4 fluorometer. Lysates was stored in-80° C. before use. 10 μg of sample was prepared with NuPAGE™ LDS Sample Buffer (Invitrogen, B0007) and NuPAGE™ Reducing Agent (Invitrogen, B0009) according to the manufacturer's instructions and run on NuPAGE™ 10% Bis-Tris Gel (Invitrogen, NP0302BOX) and Mini Gel Tank system (Invitrogen, A25977) with Bolt MES SDS Running Buffer (Invitrogen, B000202) at 100 V for 1 h. SeeBlue Plus2 Pre-Stained Protein Standard (Invitrogen, LC5925) was used as a ladder. The gel was then transferred in Mini Trans-Blot Cell system (Bio-Rad, 1703930) on to a PVDF transfer membrane (Thermo Scientific™, 88518) at 240 mA for 1 h and 30 min. The membrane was blocked with 5% BSA (GenDEPOT, A0100) in 1% TBST (Fisher Scientific, BP2471-1, BP337-100) overnight. All the primary and secondary antibodies were diluted with 5% BSA in 1% TBST. Washes were done as a short rinse followed by 5 long washes for 5 min each. Both primary (Cell Signaling Technology, 9101, 9102) and secondary antibodies (92632211, LI-COR) were incubated for 1 h at RT. The blot was imaged with Odyssey CLx (LI-COR). The blot was stripped with Restore™ PLUS Western Blot Stripping Buffer (Thermo Scientific™, 46430) for 15 min at RT, rinsed with 1% TBST and reblocked with 5% BSA for 30 min.

### Human Induced Pluripotent Stem Cell Derivation

Protocols were approved by the Northwestern University Institutional Review Boards. With informed written consent, ˜9 ml of peripheral blood was taken from each volunteer and stored at 4° C., samples were transferred to Leucosep tubes (Greiner) filled with Histopaque®-1077 (Sigma). 1×10⁶ isolated peripheral blood mononuclear cells (PMBC) were grown in 24-well tissue culture-treated plates (Greiner) in 2 ml of SFEM II (Stem Cell Technologies) supplemented with 10 ng ml⁻¹ IL3, 50 ng ml⁻¹ SCF (KITLG), 40 ng ml⁻¹ IGF1 (all Peprotech), 2 U ml⁻¹ EPO (Calbiochem), 1 μM dexamethasone (Sigma) (Chou et al., 2015). 50% medium was changed every other day. After 12 days of growth, 6×10⁴ cells were transferred to a well of a 24-well plate in 500 μL of SFEM II with growth factors supplemented with CytoTune™-iPS 2.0 Sendai Reprogramming Kit viral particle factors (Gibco™) (Fujie et al., 2014; Fusaki et al., 2009) diluted to 5% (1:20) of the manufacturer's recommendations. Cells were treated with 3.5 μL, 3.5 μl, and 2.2 μl of hKOS (0.85×10⁸ CIU ml⁻¹), hMYC (0.85×10⁸ CIU ml⁻¹), and hKLF4 (0.82×10⁸ CIU ml⁻¹), respectively at MOI of 5:5:3 (KOS:MYC:KLF4). 100% media was changed after 24 h by centrifugation (300×g, 4 min) to 2 ml fresh SFEM II with growth factors, and cells were transferred to one well of a 6-well plate (Greiner) coated with 1:800 Matrigel®. 50% medium was changed gently every other day. On day 8 after transduction, 100% of medium was changed to B8 medium. Medium was changed every day. At day 17 individual colonies were picked into a Matrigel®-coated 12-well plate (one colony per well).

### Pluripotent Cell Flow Cytometry

hiPSCs were dissociated with TrypLE™ Express (Gibco™) for 3 min at 37° C. and 1×10⁶ cells were transferred to flow cytometry tubes (Falcon, 352008). Cells were stained in 0.5% fatty acid-free albumin in DPBS using 1:20 mouse IgG3 SSEA4-488 clone MC-813-70 (R&D Systems, FAB1435F, lot. YKM0409121) and 1:20 mouse IgM TRA-1-60-488 clone TRA-1-60 (BD Biosciences, 560173, lot. 5261629) for 30 min on ice then washed. Isotype controls mouse IgG3-488 clone J606 (BD Biosciences, 563636, lot. 7128849) and mouse IgM-488 clone G155-228 (BD Bioscience, 562409, lot. 7128848) were used to establish gating. All cells were analyzed using a CytoFLEX (Beckman Coulter) with CytExpert 2.2 software.

### Immunofluorescent Staining

hiPSCs were dissociated with 0.5 mM EDTA and plated onto Matrigel®-treated Nunc Lab-Tek II 8-chamber slides in B8 medium for three days (B8T for the first 24 h). Cells were fixed, permeabilized, and stained for OCT4, SSEA4, SOX2, TRA-1-60 with PSC 4-Marker Immunocytochemistry Kit (Life Technologies, A24881, Lot. 1610720) according to the manufacturer's instructions. Cells were washed three times and mounted with ProLong™ Diamond Antifade Mountant with DAPI (Invitrogen). Slides were imaged with a Ti-E inverted fluorescent microscope (Nikon Instruments) and a Zyla sCMOS camera (Andor) using NIS-Elements 4.4 Advanced software.

### Population Doubling Level Assessment

Population doubling level (PDL) was calculated according to the following formula:

$$PDL = 3.32 [ {log}_{10} ( n / {n}_{0} ) ]$$

Where n=cell number and n₀=number of cells seeded

### Cardiac Differentiation

Differentiation into cardiomyocytes was performed according to previously described protocol with slight modifications (Burridge et al., 2015; Burridge et al., 2014). Briefly, hiPSCs were split at 1:20 ratios using 0.5 mM EDTA as above and grown in B8 medium for 4 days reaching ˜75% confluence. At the start of differentiation (day 0), B8 medium was changed to CDM3 (chemically defined medium, three components) (Burridge et al., 2014), consisting of RPMI 1640 (Corning®, 10-040-CM), 500 μg ml⁻¹ fatty acid-free bovine serum albumin (GenDEPOT), and 200 μg ml⁻¹ L-ascorbic acid 2-phosphate (Wako). For the first 24 h, CDM3 medium was supplemented with 6 μM of glycogen synthase kinase 3-β inhibitor CHIR99021 (LC Labs, C-6556). On day 1, medium was changed to CDM3 and on day 2 medium was changed to CDM3 supplemented with 2 μM of the Wnt inhibitor Wnt-C59 (Biorbyt, orb181132). Medium was then changed on day 4 and every other day for CDM3. Contracting cells were noted from day 7. On day 14 of differentiation, cardiomyocytes were dissociated using DPBS for 20 min at 37° C. followed by 1:200 Liberase TH (Roche) diluted in DPBS for 20 min at 37° C., centrifuged at 300 g for 5 min, and filtered through a 100 μm cell strainer (Falcon).

### Endothelial Differentiation

hiPSCs were grown to approximately 50-70% confluent and differentiated according to an adapted version of a protocol previously described (Patsch et al., 2015). On day 5 of differentiation, endothelial cells were dissociated with Accutase® (Gibco™) for 5 min at 37° C., centrifuged at 300 g for 5 min, and analyzed.

### Epithelial Differentiation

hiPSCs were split at 1:20 ratios using 0.5 mM EDTA as above and grown in B8T medium for 1 day reaching ˜15% confluence at the start of differentiation. Surface ectoderm differentiation was performed according to an adapted version of previously described protocols (Li et al., 2015; Qu et al., 2016). On day 4 of differentiation, epithelial cells were dissociated with Accutase (Gibco™) for 5 min at 37° C., centrifuged at 300 g for 5 min, and analyzed.

### Differentiated Cell Flow Cytometry

Cardiomyocytes were dissociated with Liberase TH as described above, transferred to flow cytometry tubes and fixed with 4% PFA (Electron Microscopy Services) for 15 min at RT, and then permeabilized with 0.1% Triton X-100 (Fisher BioReagents) for 15 min at RT, washed once with DPBS, and stained using 1:33 mouse monoclonal IgG₁ TNNT2-647 clone 13-11 (BD Biosciences, 565744, lot. 7248637) for 30 min at RT and washed again. Isotype control mouse IgG1-647 clone MOPC-21 (BD Biosciences, 565571, lot. 8107668) was used to establish gating. Endothelial cells were dissociated with Accutase® as described above, transferred to flow cytometry tubes and stained with 1:100 mouse IgG2a CD31-647 clone M89D3 (BD Bioscience, 558094, lot. 8145771) for 30 min on ice then washed once with DPBS. Isotype control mouse IgG1-647 clone MOPC-21 (BD Biosciences, 565571, lot. 8107668) was used to establish gating. Epithelial cells were dissociated with Accutase® as described above, transferred to flow cytometry tubes, fixed with 4% PFA (Electron Microscopy Services) for 10 min at RT, and then permeabilized with 0.1% saponin (Sigma) in DPBS for 15 min at RT. Cells were washed once in wash buffer (DPBS with 5% FBS, 0.1% NaN₃, 0.1% saponin), stained with 1:200 mouse IgG1 KRT18-647 clone DA-7 (BioLegend, 628404, lot. B208126) for 30 min at RT, then washed twice with wash buffer. Isotype control mouse IgG1-647 clone MOPC-21 (BD Biosciences, 565571, lot. 8107668) was used to establish gating. All cells were analyzed using a CytoFLEX (Beckman Coulter) with CytExpert 2.2 software.

### Statistical Methods

Data were analyzed in Excel or R and graphed in GraphPad Prism 8. Detailed statistical information is included in the corresponding figure legends. Data were presented as mean±SEM. Comparisons were conducted via an unpaired two-tailed Student's t-test with significant differences defined as P<0.05 (*), P<0.01 (**), P<0.005 (***), and P<0.0001 (****). No statistical methods were used to predetermine sample size. The experiments were not randomized, and the investigators were not blinded to allocation during experiments and outcome assessment.

### Example 1: Optimization of Media Constituents with a Short-Term Assay

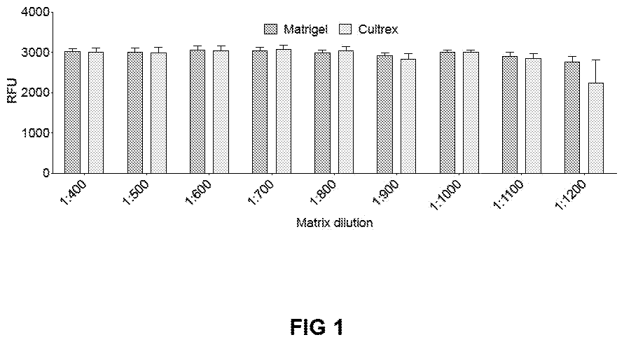

As a first step, concentrations of matrices on which hiPSCs are grown was determined. Although laminin-511 (Rodin et al., 2010), laminin-521 (Rodin et al., 2014), vitronectin (Braam et al., 2008), and Synthemax™-II (Melkoumian et al., 2010) are suitable for hiPSC culture (Burridge et al., 2014), none are appropriately cost-effective, and in the case of vitronectin or Synthemax-II, also not suitable for subsequent differentiation (Burridge et al., 2014). Matrigel®, although an undefined product (Hughes et al., 2010), is a cost-effective and commonly used matrix with substantial data using it at 50 μg cm⁻² (Ludwig et al., 2006a). Comparing two similar commercial products, Matrigel® (Corning®) and Cultrex®/Geltrex® (Trevigen/Gibco™M), we found that both be used at concentrations as low as 10 μg cm⁻² (a 1:1000dilution) (FIG. 1) and were subsequently used at a conservative 1:800 dilution for all future experiments.

A pluripotent growth assay modeled on that used by Ludwig et al. and Chen et al. was established after determining that neither automated cell counting of dissociated cells (6-well) or small format survival assays (i.e. 96-well) were suitably robust. The growth assay may be outlined as follows:

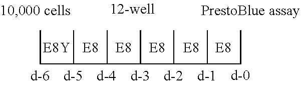

During the development of this assay platform, we found that precise measurement of relative growth was a suitable surrogate for metrics of the pluripotent state (such as NANOG expression). When cells began to spontaneously differentiate, such as when TGFβ1 is omitted from the formula, the resulting slowing of growth was easily detectable.

Each component of a commercially available cell culture media was evaluated for performance and cost effectiveness. The commercially available cell culture media comprises:

| Component | E8 | |
|---|---|---|
| DMEM/F12 | 1 | |
| Insulin | 20 | μg ml⁻¹ |
| Ascorbic acid 2-phosphate | 64 | μg ml⁻¹ |
| Transferrin | 10 | μg ml⁻¹ |
| Sodium selenite | 14 | ng ml⁻¹ |
| FGF2 | 100 | ng ml⁻¹ |
| TGFβ1 | 2 | ng ml⁻¹ |
| Sodium bicarbonate | 1743 | μg ml⁻¹ |
| pH | 7.4 | |
| Osmolarity | 340 | mOsm/l |

A range of concentrations for each of the six major hiPSC medium components (FIG. 2A-F) was assessed. During this stage we established that insulin was essential and could only be replaced by very high levels of IGF1 LR3 (≥1 μg ml⁻¹), although this was not cost-effective (FIG. 2A). The effect of insulin was dose-dependent up to 20 μg ml⁻¹. Ascorbic acid 2-phosphate was not essential, as previously demonstrated (Prowse et al., 2010), but higher levels (≥200 μg ml⁻¹) enhanced growth (FIG. 2B). Of note, this level of ascorbic acid 2-phosphate is similar to the level optimized in the cardiac differentiation media CDM3 (Burridge et al., 2014). Transferrin was also not essential to the media formula, but improved growth in a dose-dependent manner with 20 μg ml⁻¹ exhibiting optimal growth while maintaining cost-efficiency (FIG. 2C). A source of selenium was shown to be essential, although concentrations of sodium selenite between 2-200 ng ml⁻¹ did not significantly affect growth, and sodium selenite became toxic at ≥200 ng ml⁻¹ (FIG. 2D). FGF2-K128N was optimal at ≥40 ng ml⁻¹ (FIG. 2E), and TGFβ1 was sufficient at ≥0.5 ng ml⁻¹ in this simple one passage growth assay (FIG. 2F).

### Example 2: Optimization of Additional Media Components

Referring now to FIG. 3, additional media constituents in the short-term assay of Example 1 were evaluated. FIG. 3A suggests that the inclusion of a ROCK 1/2 inhibitor for at least the first 24 h after passage is optimal.

The addition of 2 μM thiazovivin for the first 24 h marginally, though not significantly, improved growth over 10 μM Y27632 and was ˜5× more cost-effective choice (FIG. 3B).

Some recent hiPSC growth formulae have suggested that the addition of high levels (2×) of non-essential amino acids (NEAA) and/or low levels (0.1×) of chemically defined lipids enhance growth (FIG. 3C). In our assay NEAA did not augment growth and the addition of lipids was inhibitory at all but the lowest levels (FIG. 3C).

Supplementation with bovine serum albumin (BSA), a common hPSC media component, did not have positive or negative effects on growth and was excluded to maintain a chemically defined formula (FIG. 3D).

The DMEM/F12 basal media of Corning® contains higher levels of sodium bicarbonate (˜29 mM or 2438 mg 1⁻¹ ) compared to DMEM/F12 from other manufacturers. Supplementation of Gibco™ DMEM/F12, which contains 14 mM of sodium bicarbonate, with 20 mM of additional sodium bicarbonate has recently been demonstrated to be advantageous to hiPSC growth rate by suppressing acidosis of the medium (Liu et al., 2018). However, the standard 29 mM of sodium bicarbonate was optimal according to FIG. 3E.

The effect of pH and osmolarity on growth were also evaluated. A pH 7.1 (FIG. 3F) and an osmolarity of 310 mOsm 1⁻¹ (FIG. 3G) were found to promote the highest growth rate. All of our experiments were optimized for 5% O₂ and 5% CO₂ without the significant cost increase from high N₂ usage or from 10% CO₂ suggested by some (Ludwig et al., 2006b), that necessitates higher levels of sodium bicarbonate.

### FGF2

In initial studies, a commercial recombinant human FGF2 was provided. This constituent accounted for >60% of the media cost. Additional FGF2 variants were then assessed. A FGF2 sequence with E. coli-optimized codon usage to enhance yield and a K128N point mutation to improve thermostability (Chen et al., 2012) was inserted into a recombinant protein production plasmid (pET-28a) utilizing dual 6xHis tags that were not cleaved during purification (FIG. 4). We subsequently generated two additional thermostable FGF2 variants: FGF1-4X (Chen et al., 2012), and FGF2-G3 (Dvorak et al., 2018). We found FGF2-G3, with nine point-mutations was more potent than FGF2-K128N, showing a similar effect on growth rate at 5 ng ml⁻¹ to FGF2-K128N at 100 ng ml⁻¹ (FIG. 3H).

### Example 3: Optimization of Media Components Using a Long-Term Assay

Our initial short-term experiments provided preliminary optimizations, yet we were aware from previous experiments that some variables, such as the elimination of TGFβ1, had minimal effects short-term and would only have detectable negative effects in long-term experiments. Thus the effectiveness of a long-term assay was evaluated with the following assay protocol:

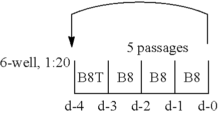

Each experiment was independently repeated at least 5 times. These experiments again confirmed concentrations of insulin (20 μg ml⁻¹; FIG. 5A), ascorbic acid 2-phosphate (200 μg ml−1; FIG. 5B), transferrin (20 μg ml⁻¹; FIG. 5C), sodium selenite (20 ng ml⁻¹; FIG. 5D), and FGF2-G3 (40 ng ml⁻¹; FIG. 5E).

As TGFβ1 is now the costliest component (˜20% of total cost) we found that this could be used at lower concentrations than previously suggested (0.1 ng ml⁻¹) even in a long-term assay (FIG. 5F). We found that other Activin/NODAL/TGFβ1 signaling sources such as NODAL were required at cost-prohibitively high levels (100 ng ml⁻¹) (FIG. 5G), and Activin A was not suitable to maintain growth to the same level as TGFβ1 (FIG. 5H). Activin A in combination with TGFβ1 also has had a negative effect on growth (FIG. 5I). TGFβ1 is a homodimer of two TGFB1 gene products and therefore the recombinant protein is commonly produced in mammalian cells making it complex to produce for basic research labs. These mammalian cells provide post-translational modifications crucial for biological activity such as glycosylation, phosphorylation, proteolytic processing, and formation of disulfide bonds which are not present in E. coli. To overcome this issue, we generated a version of TGFβ1 (TGFβ1m) that is unable to form dimers due to a point mutation. This monomeric protein is predicted to be ˜20-fold less potent than TGFβ1 but can be easily produced in large quantities in E. coli (Kim et al., 2015). Our initial experiments demonstrated the TGFB1 sequence with E. coli-optimized codon usage was expressed in inclusion bodies. To overcome this, we inserted the sequence into a protein production plasmid (pET-32a) designed to produce a thioredoxin fusion protein allowing expression in the cytoplasm. It has been also demonstrated that TGFβ3 is more potent than TGFβ1 (Huang et al., 2014). Comparing TGFβ1, TGFβ1m, TGFβ3, and TGFβ3m, we found that TGFβ3 offered the best combination of being able to be produced in E. coli and suitable for use at 0.1 ng ml⁻¹ (FIG. 5J-L) and was therefore selected for the final formula.

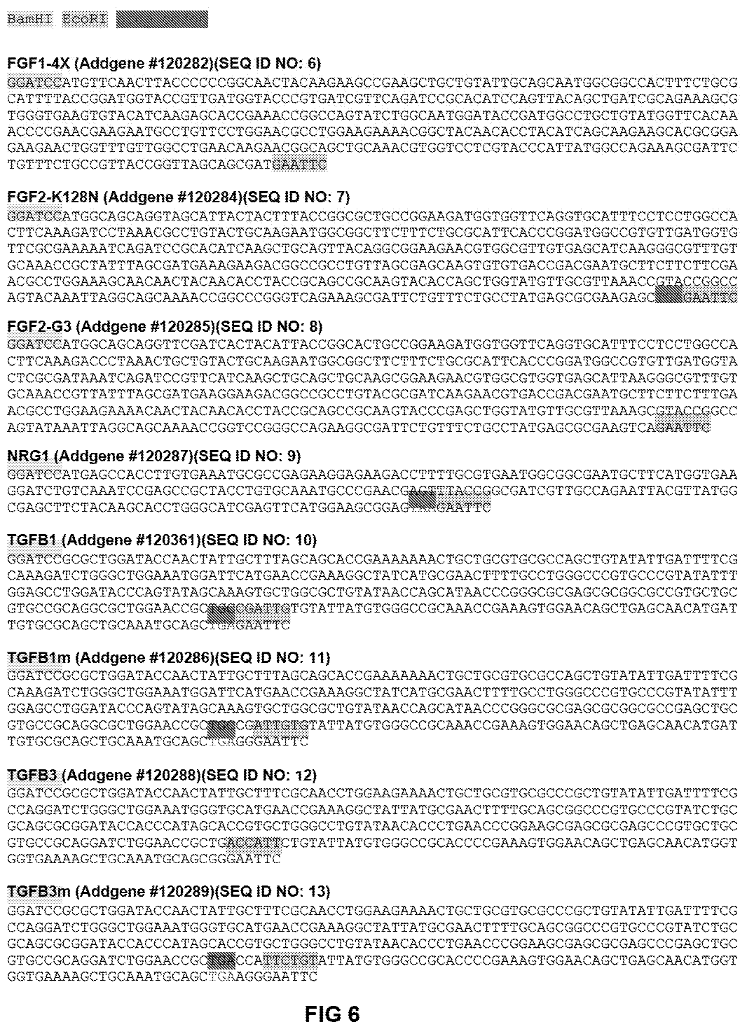

Finally, two hESC media formulations that we studied, DC-HAIF and iDEAL, contain both FGF2 and neuregulin 1 (NRG1). We found that supplementation with all tested levels of NRG1 enhanced growth by >15% over FGF2-G3 alone (FIG. 5M-N), although NRG1 is not able to support growth in the absence of FGF2 (FIG. 5O). We similarly generated recombinant NRG1 protein using pET-32a, which prevented production as inclusion bodies, with subsequent cleavage using thrombin to form an active protein (FIG. 5P and FIG. 6).

One final B8 media formulation of the invention derived from these long-term assay optimizations is:

| Component | B8 | |
|---|---|---|
| DMEM/F12 | 1 | |
| Insulin | 20 | μg ml⁻¹ |
| Ascorbic acid 2-phosphate | 200 | μg ml⁻¹ |
| Transferrin | 20 | μg ml⁻¹ |
| Sodium selenite | 20 | ng ml⁻¹ |
| FGF2-G3 | 40 | ng ml⁻¹ |
| TGFβ3 | 0.1 | ng ml⁻¹ |
| NRG1 | 0.1 | ng ml⁻¹ |
| Sodium bicarbonate | 2438 | μg ml⁻¹ |
| pH | 7.1 | |
| Osmolarity | 310 | mOsm/l |

### Example 4: Demonstration of the Suitability of B8 for hiPSC Generation

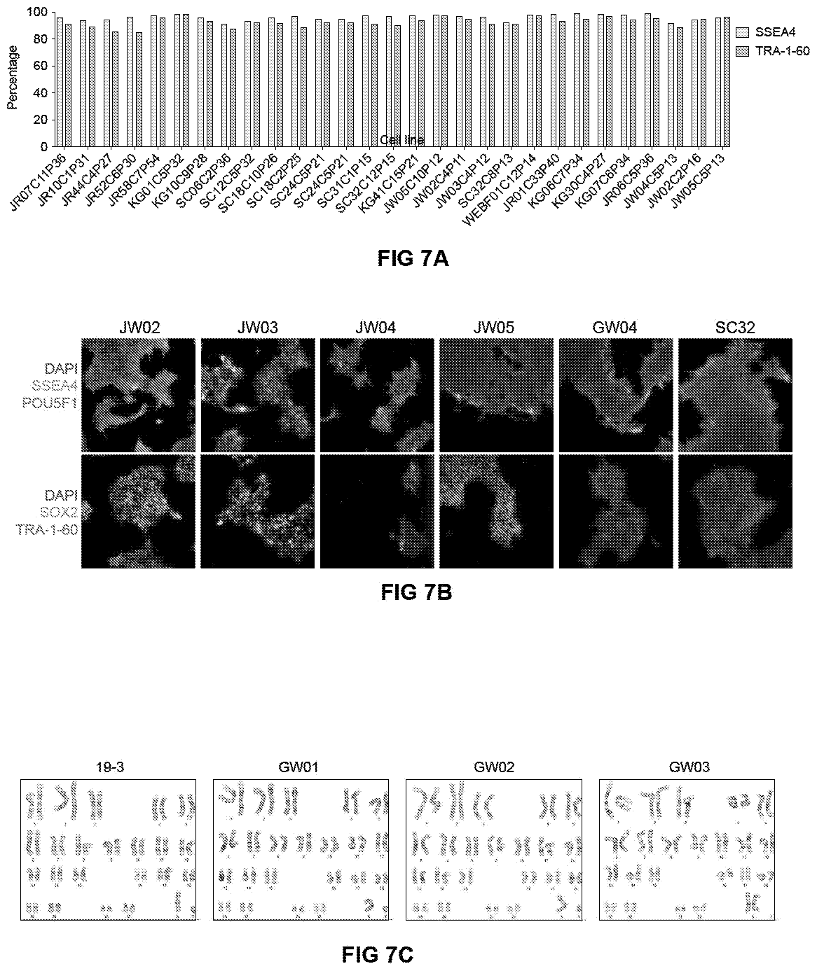

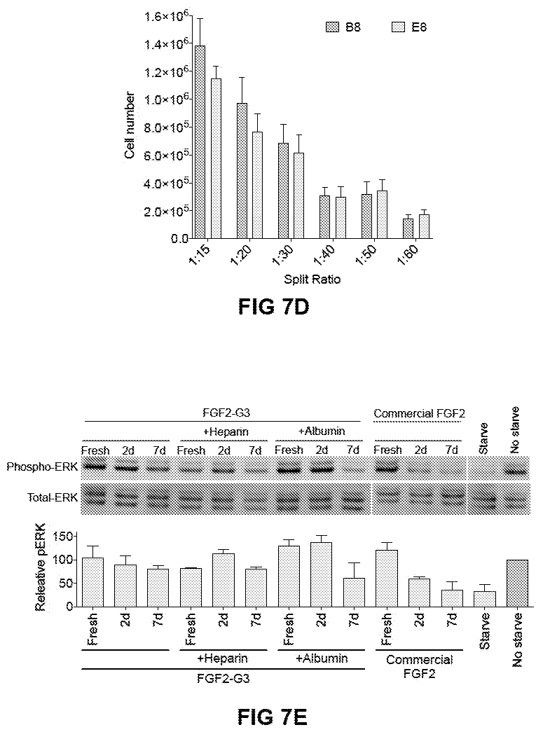

The suitability of the B9 media to generate hiPSC lines was confirmed. We generated hiPSC lines from 26 patients using established protocols but using B8. Lines were successfully generated from all donors and passed standard assays for pluripotency including flow cytometry for SSEA4 and TRA-1-60 (FIG. 7A), immunofluorescent staining for SSEA4, POU5F1, SOX2, and TRA-1-60 (FIG. 7B), and karyotype stability (FIG. 7C). We then compared this B8 formula with commercially available E8 formula and found that growth similar to B8 across commonly used split ratios (FIG. 7D). It has been demonstrated that unlike commercial FGF2, thermostable variants of FGF2 such as FGF2-G3 are capable of inducing pERK in FGF-starved cells, even after media had previously been stored for extended periods at 37° C. (Chen et al., 2012; Dvorak et al., 2018). To confirm that our FGF2-G3 performed similarly we performed a comparable assay and corroborated that FGF2-G3 was stable after 7 days at 37° C., whereas commercial FGF2 was not capable of stimulating pERK after 2 days at 37° C. As common ‘weekend-free’ media formulae such as StemFlex have reverted to using albumin and likely contain heparin, both of which are not chemically defined, we also assessed the function of these components in this assay and found that both improved the stability of FGF2-G3 (FIG. 7E).

### Example 5: Demonstration of suitability of B8 for Weekend-Free Culture

With the understanding that B8 supports hiPSC growth across a variety of sub-optimal conditions (FIG. 7D), we established the suitability of this formula to skip days of media change. We established a matrix of media change days both with and without thiazovivin (FIG. 8A) and showed that daily B8 media changes (top row) was surprisingly the least suitable for growth rate. B8T treatment without media changes was the least effective of the thiazovivin-containing protocols (bottom row), whereas B8T followed by B8 (dark grey bar) was a suitable compromise between growth rate and extended exposure to thiazovivin. With the knowledge of the influence of various media change-skipping timelines, we devised a 7-day schedule consisting of passaging cells every 3.5 days that would allow for the skipping of media changes on the weekends, a major caveat of current hiPSC culture protocols (FIGS. 8B and 8C). Our data demonstrated that a 3.5-day schedule with medium exchange after the first 24 h was suitable long-term, whereas no media exchange (i.e. passage only) was not suitable (FIG. 8D). In addition, supplementation of the media with 0.5 mg ml⁻¹ of albumin did not rescue this deficit (FIG. 8D). Further experiments with various doses of albumin or heparin further confirmed that these do not have an additive effect in B8 (FIG. 8E). One of the major caveats of skipping media changes is that differentiation efficiency and reproducibility is diminished. We used our existing cardiac (Burridge et al., 2014), endothelial (Patsch et al., 2015), and epithelial differentiation (Li et al., 2015; Qu et al., 2016) protocols to demonstrated that B8 supported differentiation to a similarly high level with either daily media change or our 7-day protocol (FIGS. 8F and 8G).

As will culture of cells in any media for an extended period of time, other factors should be considered. For example, we know that the L-glutamine in the medium is unstable at 37° C. and that the concentration is reduced by about a third over four days. We also know that cells are producing ammonia, reducing the pH of the media, although this buffered partially by the HEPES and sodium bicarbonate. Finally, hiPSCs release autocrine or paracrine factors into the media that may induce differentiation and the increase in these factors over time has not been decoupled from the use of nutrients and production of metabolic waste.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of and “consisting of” those certain elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. See In re Herz, 537 F.2d 549, 551-52, 190 U.S.P.Q. 461, 463 (CCPA 1976) (emphasis in the original); see also MPEP § 2111.03. Thus, the term “consisting essentially of” should not be interpreted as equivalent to “comprising.” Moreover, the present disclosure contemplates that in some embodiments, any feature or combination of features can be excluded or omitted.

To illustrate, if the specification states that a complex comprises components A, B, and C, it is specifically intended that any of A, B, or C, or a combination thereof, can be omitted and disclaimed singularly or in any combination. Recitation of ranges of values are merely intended to serve as a shorthand method of referring individually to each separate value falling within the range, unless otherwise stated, and each separate value is incorporated into the specification as if it were individually recited. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Unless otherwise defined, all technical terms have the same meaning as commonly understood by one of ordinary skill in the art to which this disclosure belongs.

As stated above, while the present application has been illustrated by the description of embodiments, and while the embodiments have been described in considerable detail, it is not the intention to restrict or in any way limit the scope of the appended claims to such detail. Additional advantages and modifications will readily appear to those skilled in the art, having the benefit of this application. Therefore, the application, in its broader aspects, is not limited to the specific details and illustrative examples shown. Departures may be made from such details and examples without departing from the spirit or scope of the general inventive concept.

1-18. (canceled)

19. A method of expanding a pluripotent cell population, the method comprising culturing the cell population on a surface contacting a chemically defined liquid cell culture medium a first time for at least 2 days, passaging the cell population, culturing the cell population on the surface contacting the chemically defined liquid cell culture medium a second time for at least 2 days, and subjecting the cell population to a monolayer differentiation protocol, wherein the chemically defined liquid cell culture medium comprises a nonzero amount of TGF beta less than 2 ng/ml and a nonzero amount of FGF2 less than 100 ng/ml.

20. The method of claim 19, comprising repeating the culturing the cell population on a surface contacting a chemically defined liquid cell culture medium for at least 5 times for at least 2 days, each of the times punctuated by passaging the cell population.

21. The method of claim 20, comprising repeating the culturing the cell population on a surface contacting a chemically defined liquid cell culture medium for at least 10 times for at least 2 days, each of the times punctuated by passaging the cell population.

22. The method of claim 20, comprising repeating the culturing the cell population on a surface contacting a chemically defined liquid cell culture medium for at least 50 times for at least 2 days, each of the times punctuated by passaging the cell population.

23. The method of claim 19, wherein the cell population exhibits at least 3 population doublings per cell passage.

24. The method of claim 19, wherein the cell population is passaged no more than twice per week.

25. The method of claim 19, wherein culturing the cell population on a surface contacting a chemically defined liquid cell culture medium comprises depositing the cell population at a confluence of less than 5%, and observing monolayer growth of at least one doubling per day.

26. The method of claim 19, comprising observing at least 70% of the cell population to differentiate.

27. The method of claim 19, wherein the cell population exhibits at least 80% maintenance of a pluripotency marker prior to inducing the cell population to differentiate.

28. The method of claim 27, wherein the pluripotency marker is selected from the list consisting of flow cytometry detection of SSEA4, flow cytometry detection of TRA1-60, immunofluorescent staining for SSEA4, immunofluorescent staining for POU5F1, immunofluorescent staining for SOX2, and immunofluorescent staining for TRA-1-60, karyotype stability, and pluripotency marker morphology.

29. The method of claim 19, wherein the TGF beta comprises at least one of TGF beta 1 or TGF beta 3.

30. The method of claim 19, wherein the FGF2 comprises FGF2-G3.

31. The method of claim 19, wherein the FGF2 is thermostable at 37 C for at least 2 days in culturing conditions.

32. The method of claim 19, wherein the chemically defined liquid cell culture medium comprises a basal medium comprising salts, amino acids, and vitamins, insulin, ascorbic acid 2-phosphate, transferrin, selenite, NRG, and sodium bicarbonate.

33. The method of claim 32, wherein the basal medium comprises DMEM/F12.

34. The method of claim 32, wherein the chemically defined liquid cell culture medium does not comprise Activin A at a concentration sufficient to influence cell growth, differentiation, or health.

35. The method of claim 32, wherein the chemically defined liquid cell culture medium comprises at least one reagent selected from the list consisting of NODAL and NRG1.

36. The method of claim 19, wherein culturing the cell population on the surface contacting the chemically defined liquid cell culture medium comprises changing the medium at least one during the at least 2 days.

37. The method of claim 36, wherein the chemically defined liquid cell culture medium comprises less than 1 ng/ml TGF beta and less than 1 ng/ml NRG1.

38. The method of claim 32, wherein the chemically defined liquid cell culture medium comprises no other cell growth factor at a concentration sufficient to influence cell growth, differentiation, or health.


SEQUENCE LISTING (17 sequences)

SEQ ID NO: 1 - protein, 141 aa, synthetic construct
  REGION 1..141: note = Synthetic
MFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 2 - protein, 141 aa, synthetic construct
  REGION 1..141: note = Synthetic
MFNLPPGNYKKPKLLYCSNGGHFLRILPDGTVDGTRDRSDPHIQLQLIAESVGEVYIKSTETGQYLAMDTDGLLYGSQTPNEECLFLERLEENGYNTYISKKHAEKNWFVGLNKNGSCKRGPRTHYGQKAILFLPLPVSSD

SEQ ID NO: 3 - protein, 155 aa, synthetic construct [sequence omitted]

SEQ ID NO: 4 - protein, 155 aa, synthetic construct
  REGION 1..155: note = Synthetic
MAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALNRTGQYKLGSKTGPGQKAILFLPMSAKS

SEQ ID NO: 5 - protein, 155 aa, synthetic construct
  REGION 1..155: note = Synthetic
MAAGSITTLPALPEDGGSGAFPPGHFKDPKLLYCKNGGFFLRIHPDGRVDGTRDKSDPFIKLQLQAEERGVVSIKGVCANRYLAMKEDGRLYAIKNVTDECFFFERLEENNYNTYRSRKYPSWYVALKRTGQYKLGSKTGPGQKAILFLPMSAKS

SEQ ID NO: 6 - DNA, 435 bp, synthetic construct
  misc_feature 1..435: note = Synthetic
  source 1..435: mol_type = other DNA
ggatccatgttcaacttaccccccggcaactacaagaagccgaagctgctgtattgcagcaatggcggccactttctgcgcattttaccggatggtaccgttgatggtacccgtgatcgttcagatccgcacatccagttacagctgatcgcagaaagcgtgggtgaagtgtacatcaagagcaccgaaaccggccagtatctggcaatggataccgatggcctgctgtatggttcacaaaccccgaacgaagaatgcctgttcctggaacgcctggaagaaaacggctacaacacctacatcagcaagaagcacgcggagaagaactggtttgttggcctgaacaagaacggcagctgcaaacgtggtcctcgtacccattatggccagaaagcgattctgtttctgccgttaccggttagcagcgatgaattc

SEQ ID NO: 7 - DNA, 480 bp, synthetic construct
  misc_feature 1..480: note = Synthetic
  source 1..480: mol_type = other DNA
ggatccatggcagcaggtagcattactactttaccggcgctgccggaagatggtggttcaggtgcatttcctcctggccacttcaaagatcctaaacgcctgtactgcaagaatggcggcttctttctgcgcattcacccggatggccgtgttgatggtgttcgcgaaaaatcagatccgcacatcaagctgcagttacaggcggaagaacgtggcgttgtgagcatcaagggcgtttgtgcaaaccgctatttagcgatgaaagaagacggccgcctgttagcgagcaagtgtgtgaccgacgaatgcttcttcttcgaacgcctggaaagcaacaactacaacacctaccgcagccgcaagtacaccagctggtatgttgcgttaaaccgtaccggccagtacaaattaggcagcaaaaccggcccgggtcagaaagcgattctgtttctgcctatgagcgcgaagagctgagaattc

SEQ ID NO: 8 - DNA, 477 bp, synthetic construct
  misc_feature 1..477: note = Synthetic
  source 1..477: mol_type = other DNA
ggatccatggcagcaggttcgatcactacattaccggcactgccggaagatggtggttcaggtgcatttcctcctggccacttcaaagaccctaaactgctgtactgcaagaatggcggcttctttctgcgcattcacccggatggccgtgttgatggtactcgcgataaatcagatccgttcatcaagctgcagctgcaagcggaagaacgtggcgtggtgagcattaagggcgtttgtgcaaaccgttatttagcgatgaaggaagacggccgcctgtacgcgatcaagaacgtgaccgacgaatgcttcttctttgaacgcctggaagaaaacaactacaacacctaccgcagccgcaagtacccgagctggtatgttgcgttaaagcgtaccggccagtataaattaggcagcaaaaccggtccgggccagaaggcgattctgtttctgcctatgagcgcgaagtcagaattc

SEQ ID NO: 9 - DNA, 213 bp, synthetic construct
  misc_feature 1..213: note = Synthetic
  source 1..213: mol_type = other DNA
ggatccatgagccaccttgtgaaatgcgccgagaaggagaagaccttttgcgtgaatggcggcgaatgcttcatggtgaaggatctgtcaaatccgagccgctacctgtgcaaatgcccgaacgagtttaccggcgatcgttgccagaattacgttatggcgagcttctacaagcacctgggcatcgagttcatggaagcggagtaagaattc

SEQ ID NO: 10 - DNA, 351 bp, synthetic construct
  misc_feature 1..351: note = Synthetic
  source 1..351: mol_type = other DNA
ggatccgcgctggataccaactattgctttagcagcaccgaaaaaaactgctgcgtgcgccagctgtatattgattttcgcaaagatctgggctggaaatggattcatgaaccgaaaggctatcatgcgaacttttgcctgggcccgtgcccgtatatttggagcctggatacccagtatagcaaagtgctggcgctgtataaccagcataacccgggcgcgagcgcggcgccgtgctgcgtgccgcaggcgctggaaccgctgccgattgtgtattatgtgggccgcaaaccgaaagtggaacagctgagcaacatgattgtgcgcagctgcaaatgcagctgagaattc

SEQ ID NO: 11 - DNA, 277 bp, synthetic construct
  misc_feature 1..277: note = Synthetic
  source 1..277: mol_type = other DNA
ggatccgcgctggataccaactattgctttagcagcaccgaaaaaaactgctgcgtgcgccagctgtatattgattttcgcaaagatctgggctggaaatggattcatgaaccgaaaggctatcatgcgaacttttgcctgggcccgtgcccgtatatttggagcctggatacccagtatagcaaagtgctggcgctgtataaccagcataacccgggcgcgagcgcggcgccgagctgcgtgccgcaggcgctggaaccgctgccgattgtgtatt

SEQ ID NO: 12 - DNA, 273 bp, synthetic construct
  misc_feature 1..273: note = Synthetic
  source 1..273: mol_type = other DNA
ggatccgcgctggataccaactattgctttcgcaacctggaagaaaactgctgcgtgcgcccgctgtatattgattttcgccaggatctgggctggaaatgggtgcatgaaccgaaaggctattatgcgaacttttgcagcggcccgtgcccgtatctgcgcagcgcggataccacccatagcaccgtgctgggcctgtataacaccctgaacccggaagcgagcgcgagcccgtgctgcgtgccgcaggatctggaaccgctgaccattctg

SEQ ID NO: 13 - DNA, 354 bp, synthetic construct
  misc_feature 1..354: note = Synthetic
  source 1..354: mol_type = other DNA
ggatccgcgctggataccaactattgctttcgcaacctggaagaaaactgctgcgtgcgcccgctgtatattgattttcgccaggatctgggctggaaatgggtgcatgaaccgaaaggctattatgcgaacttttgcagcggcccgtgcccgtatctgcgcagcgcggataccacccatagcaccgtgctgggcctgtataacaccctgaacccggaagcgagcgcgagcccgagctgcgtgccgcaggatctggaaccgctgaccattctgtattatgtgggccgcaccccgaaagtggaacagctgagcaacatggtggtgaaaagctgcaaatgcagctgaagggaattc

SEQ ID NO: 14 - protein, 154 aa, synthetic construct
  REGION 1..154: note = Synthetic
AAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 15 - protein, 154 aa, synthetic construct
  REGION 1..154: note = Synthetic
AAGSITTLPALPEDGGSGAFPPGHFKDPKLLYCKNGGFFLRIHPDGRVDGTRDKSDPFIKLQLQAEERGVVSIKGVCANRYLAMKEDGRLYAIKNVTDECFFFERLEENNYNTYRSRKYPSWYVALKRTGQYKLGSKTGPGQKAILFLPMSAKS

SEQ ID NO: 16 - protein, 112 aa, synthetic construct [sequence omitted]

SEQ ID NO: 17 - protein, 65 aa, synthetic construct
  REGION 1..65: note = Synthetic
SHLVKCAEKEKTFCVNGGECFMVKDLSNPSRYLCKCPNEFTGDRCQNYVMASFYKHLGIEFMEAE